(19) United States
(12) Patent Application Publication
(10) Pub. No.: US 2008/0207726 A1
(43) Pub. Date: Aug. 28, 2008

(54) Process for the purification of carvedilol or its salts thereof

(76) Inventors: Santiago Ini, Haifa (IL); Ron Bar-Shavit, Ariel (IL); Marina Isaev, Hertzlya (IL)

Correspondence Address: KENYON & KENYON LLP, ONE BROADWAY, NEW YORK, NY, 10004, US

(21) Appl. No.: 11/823,910

(22) Filed: Jun. 28, 2007

Related U.S. Application Data
(60) Provisional application No. 60/932,205, filed on May 29, 2007.
(60) Provisional application No. 60/903,696, filed on Feb. 26, 2007.
(60) Provisional application No. 60/927,098, filed on Apr. 30, 2007.

(51) Int. Cl. A61K 31/403; A61P 9/00; C07D 209/84

(52) U.S. Cl. 514/411; 548/444

(57) ABSTRACT

The present invention provides processes for reducing bis impurities ((1,1′-[2-(2-methoxyphenoxy)ethyl]iminobis-[3-(9H-carbazol-4-yloxy)]-propan-2-ol)), in particular Bis 1 and Bis 2, in carvedilol preparations. The process may comprise (a) combining carvedilol base with phosphoric acid in ethanol to obtain a reaction mixture; and (b) precipitating carvedilol phosphate from the reaction mixture, where the carvedilol phosphate comprises low levels of Bis 1 and Bis 2.

### CROSS REFERENCE TO RELATED APPLICATIONS

This application claims the benefit of U.S. Provisional Patent Application No. 60/932,205 filed 29 May 2007 as attorney docket no. 1662/A440P1, the contents of which are incorporated herein by reference.

### FIELD OF THE INVENTION

The present invention describes a process for the purification of carvedilol and salts thereof.

### BACKGROUND OF THE INVENTION

Carvedilol, (±)-1-(Carbazol-4-yloxy)-3-[[2-(o-methoxyphenoxy)ethyl]amino]-2-propanol, is a nonselective β-adrenergic blocker with α₁-blocking activity. carvedilol is a racemic mixture having the following structural formula:

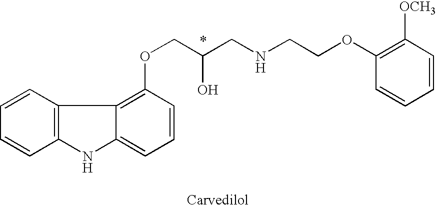

Carvedilol and carvedilol phosphate are the active ingredients in COREG® and COREG CR® respectively. Both compounds are indicated for the treatment of congestive heart failure and for the management of hypertension. Since carvedilol is a multiple-action drug, its beta-blocking activity affects the response to certain nerve impulses in parts of the body. As a result, beta-blockers decrease the heart's need for blood and oxygen by reducing its workload. Carvedilol is also known to be a vasodilator resulting primarily from alpha-adrenoceptor blockade. The multiple actions of carvedilol are responsible for the antihypertensive efficacy of the drug and for its effectiveness in managing congestive heart failure.

U.S. Pat. No. 4,503,067 (“'067 patent”) discloses a class of carbazolyl-(4)-oxypropanolamine compounds, including carvedilol. See '067 patent, col. 1, 1. 15 to col. 2, 1. 3. The '067 patent also discloses the conversion of the compounds in their pharmacologically acceptable salts, by reacting the compound with “an equivalent amount of an inorganic or organic acid.” See id. at col. 4, 11. 23-29.

According to EP 918055, the process described in '067 patent, results in the formation of a bis impurity (1,1′-[2-(2-methoxyphenoxy)ethyl]iminobis-[3-(9H-carbazol-4-yloxy)]-propan-2-ol):

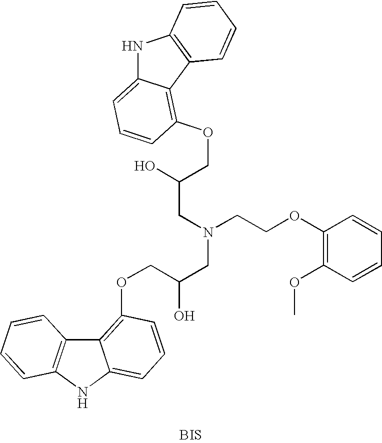

Bis is a mixture of diastereomers. Therefore under non-chiral chromatographic determination methods, two peaks are observed (Bis 1 and Bis 2).

This impurity may remain in the final product; therefore there is a need in the art for the purification of carvedilol by removing this impurity.

### SUMMARY OF THE INVENTION

The present invention provides a process for reducing bis impurities ((1,1′-[2-(2-methoxyphenoxy)ethyl]iminobis-[3-(9H-carbazol-4-yloxy)]-propan-2-ol)) in carvedilol preparations. In particular, the invention provides a process for reducing the amounts of Bis 1 and Bis 2 in carvedilol preparations. In certain embodiments, the invention provides a process comprising (a) combining carvedilol base with phosphoric acid in ethanol to obtain a reaction mixture; and (b) precipitating carvedilol phosphate from the reaction mixture, where the carvedilol phosphate comprises low levels of Bis 1 and Bis 2.

In certain embodiments, the processes disclosed herein comprise combining carvedilol base with ethanol, heating, adding phosphoric acid, and cooling.

In certain embodiments, the carvedilol preparation with reduced levels of Bis 1 and Bis 2 provided by the processes of the invention is a preparation of carvedilol phosphate. In certain embodiments, the carvedilol phosphate preparation with reduced levels of Bis 1 and Bis 2 is reacted with a base to provide carvedilol base with reduced levels of Bis 1 and Bis 2.

### BRIEF DESCRIPTION OF THE DRAWINGS

FIG. 1 shows typical results from a successful system suitability test of an HPLC system for resolving carvedilol, Bis 1, and Bis 2.

FIG. 2 shows the results of a typical HPLC run on a sample containing carvedilol phosphate, Bis 1, and Bis 2.

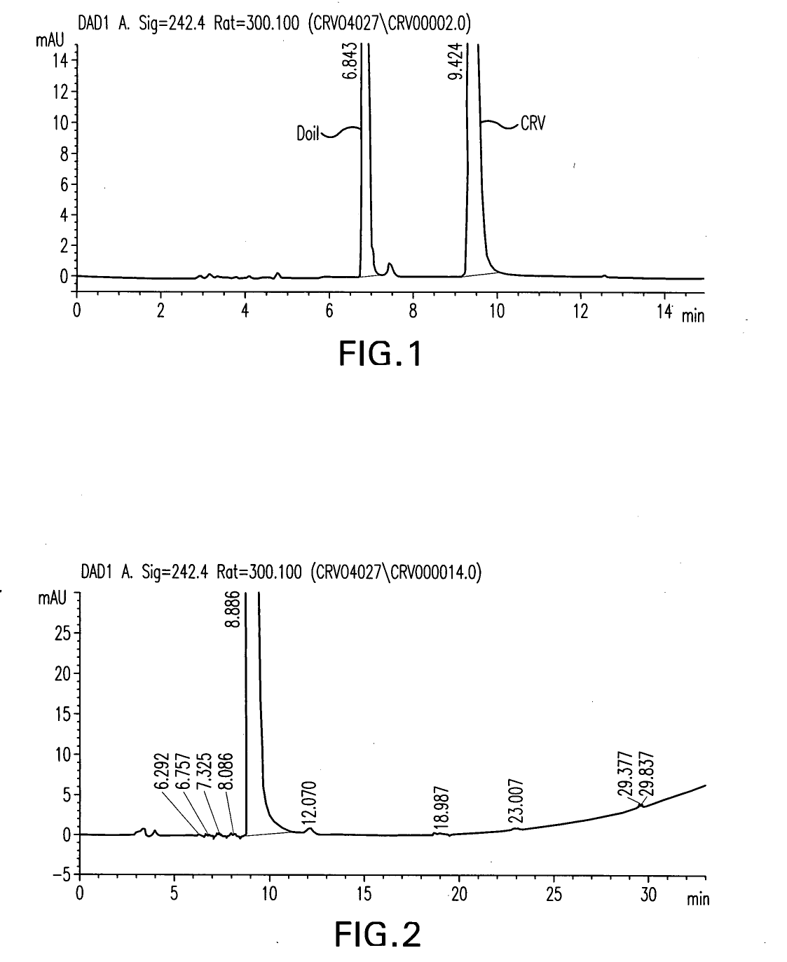

### DETAILED DESCRIPTION OF THE INVENTION

As used herein the term “bis impurity” refers to a mixture of diastereomers: Bis 1 and Bis 2, of 1,1′-[2-(2-methoxyphenoxy)ethyl]iminobis-[3-(9H-carbazol-4-yloxy)]-propan-2-ol.

As used herein, the term “carvedilol dihydrogen phosphate” refers to a carvedilol phosphate salt, in which carvedilol and phosphate are present in a molar ratio of about 1:1.

As used herein, the term “carvedilol hydrogen phosphate” refers to a carvedilol phosphate salt, in which carvedilol and phosphate are present in a molar ratio of about 2:1.

As used herein, the term “carvedilol phosphate” refers to any carvedilol phosphate salt.

Disclosed herein is a process for reducing the amount of Bis impurities in carvedilol. This process is suitable for use on an industrial scale and does not require any additional purification method such chromatography purification.

In one embodiment, the invention encompasses a process for reducing the amount of Bis impurities in carvedilol comprising reacting carvedilol base with phosphoric acid in ethanol to obtain pure carvedilol phosphate.

Preferably, the process of the invention comprises: combining carvedilol base with ethanol, preferably absolute ethanol, heating, adding phosphoric acid, and cooling, preferably in that order.

Heating can be carried out from about room temperature to about reflux temperature. Preferably, heating is carried out to obtain a solution. Preferably the temperature is about 60° C. to about 80° C., more preferably about 70° C. to about 80° C. When the desired temperature is reached, phosphoric acid is added and the mixture formed by the carvedilol, ethanol, and phosphoric acid is stirred at the desired temperature for a suitable period of time, e.g., about 2 to about 30 hours, preferably about 10 to about 24 hours, and even more preferably about 17 to about 22 hours.

After heating, the solution is cooled. Cooling is preferably carried out to a temperature to obtain a sufficient yield of a solid. Preferably cooling is carried out to less than about 40° C., preferably of about 0° C. to about 40° C. As exemplified, cooling is carried out to about 10° C. to about 30° C., preferably to about 15° C. to about 25° C., and more preferably to about 15° C. In one embodiment, cooling is carried out while stirring. Stirring can be carried out for about 1 to about 8 hours, preferably about 1 to about 4 hours, and even more preferably about 1.5 to about 3 hours. In particular, stirring can be carried out for about 1, 2, 3, 4, or more hours. Preferably, stirring is carried out for about 2 hours.

The obtained pure product can then be recovered. In one embodiment the recovery comprises: filtering, washing and drying. Preferably, the washing is with ethanol, most preferably absolute ethanol. Drying is carried out at a temperature of about 25° C. to about 100° C., preferably about 50° C. to about 60° C., under atmospheric or reduced pressure. Preferably, the drying is at a temperature of about 55° C. under vacuum (pressure of less than about 100 mmHg).

Preferably, the obtained carvedilol phosphate contains less than about 0.5% (w/w) of Bis 1, more preferably less than about 0.03% (w/w) of Bis 1; less than about 0.5% (w/w) of Bis 2, more preferably and less than about 0.03% (w/w) of Bis 2; as measured by HPLC. Preferably there is at least about 50% reduction in the amount of the Bis impurities, more preferably about a 100% reduction, relative to the amount of Bis impurities in the starting material. Preferably there is a reduction in the amount of the Bis impurities of about 60% to about 100%, more preferably about 80% to about 100%, and even more preferably about 95% to about 100%.

Preferably, the carvedilol free base employed in the present process is obtained by one of the following: (a) reaction of 4-(oxiranylmethoxy)-9H carbazole with 2-[2′-(methyoxy)phenoxy]ethylamine, or (b) reaction of 4-(oxiranylmethoxy)-9H carbazole with a 2-[2′-(methoxy)phenoxy]ethylhalide or (c) reaction of 4-(oxiranylmethoxy)-9H carbazole with a 2-[2′-(methoxy)phenoxy]sulfonate. More preferably, the carvedilol free base is obtained by reaction of 4-(oxiranylmethoxy)-9H carbazole with 2-[2′-(methyoxy)phenoxy]ethylamine.

The carvedilol phosphate obtained can be converted to carvedilol base or a pharmaceutically acceptable salt thereof. An additional step of reacting carvedilol phosphate with a base results in pure carvedilol base. Any suitable base, preferable, inorganic base such as sodium or potassium, hydroxide or carbonate, more preferably sodium bicarbonate may be used.

Pharmaceutical compositions of such carvedilol phosphate, carvedilol hydrogen phosphate, or carvedilol dihydrogen phosphate are also provided by the present invention and can be prepared by adding or mixing the carvedilol phosphate with at least one pharmaceutically acceptable excipient. “Pharmaceutically acceptable excipient” means an excipient which is not biologically or otherwise undesirable, i.e., the excipient can be administered to an individual without causing significant undesirable effects.

Pharmaceutical compositions comprising pure carvedilol base containing less than about 0.03% (w/w) of Bis 1 and less than about 0.03% (w/w) of Bis 2 as measured by HPLC, as well as methods of making such pharmaceutical compositions, are also provided.

Excipients are added to the composition for a variety of purposes. Diluents increase the bulk of a solid pharmaceutical composition and can make a pharmaceutical dosage form containing the composition easier for the patient and caregiver to handle. Diluents for solid compositions include, for example, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol and talc.

Solid pharmaceutical compositions that are compacted into a dosage form like a tablet can include excipients whose functions include helping to bind the active ingredient and other excipients together after compression. Binders for solid pharmaceutical compositions include at least one of acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate, or starch.

The dissolution rate of a compacted solid pharmaceutical composition in the patient's stomach can be increased by the addition of a disintegrant to the composition. Disintegrants include, but are not limited to, alginic acid, carboxymethylcellulose calcium, carboxymethylcellulose sodium (e.g. AC-DI-SOL @PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®) or starch.

Glidants can be added to improve the flow properties of non-compacted solid composition and improve the accuracy of dosing. Excipients that can function as glidants include colloidal silicon dioxide, magnesium trisilicate, powdered cellulose, starch, talc, and/or tribasic calcium phosphate.

When a dosage form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product form the dye. Lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and/or zinc stearate.

Flavoring agents and flavor enhancers make the dosage form more palatable to the patient. Common flavoring agents and flavor enhancers for pharmaceutical products that can be included in the composition of the present invention include, but are not limited to, maltol, vanillin, ethyl vanillin, menthol, citric acid, fumaric acid, ethyl maltol, or tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, the carvedilol described herein and any other solid excipients are dissolved or suspended in a liquid carrier such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, or cetyl alcohol.

Liquid pharmaceutical compositions of the present invention can also contain a viscosity-enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch, tragacanth or xanthan gum.

Sweetening agents such as sorbitol, saccharin, sodium saccharin, sucrose, aspartame, fructose, mannitol and/or invert sugar can be added to improve the taste.

Preservatives and chelating agents such as alcohol, sodium benzoate, butylated hydroxy toluene, butylated hydroxyanisole and ethylenediamine tetraacetic acid can be added at levels safe for ingestion to improve storage stability.

A liquid composition according to the invention can also contain a buffer such as gluconic acid, lactic acid, citric acid or acetic acid, sodium gluconate, sodium lactate, sodium citrate or sodium acetate.

Selection of excipients and the amounts to use can be readily determined by the formulation scientist based upon experience and consideration of standard procedures and reference works in the field.

The solid compositions of the invention include powders, granulates, aggregates and compacted compositions.

The carvedilol of the invention can be administered for treatment of congestive heart failure and hypertension (by any means that delivers the active ingredients) to the site of the body where beta-blocking activity exerts a therapeutic effect on the patient. For example, administration can be oral, buccal, parenteral (including subcutaneous, intramuscular, and intravenous) rectal, inhalant or ophthalmic. Although the most suitable route in any given case will depend on the nature and severity of the condition being treated, the most preferred route of the invention is oral. Carvedilol phosphate of the invention can be conveniently administered to a patient in oral unit dosage form and prepared by any of the methods well-known in the pharmaceutical arts. Dosage forms include solid dosage forms like tablets, powders, capsules, sachets, troches, or lozenges as well as liquid syrups, suspensions, or elixirs.

The active ingredient(s) and excipients can be formulated into compositions and dosage forms according to methods known in the art.

A composition for tableting or capsule filling can be prepared by wet granulation. In wet granulation some or all of the active ingredients and excipients in powder form are blended and then further mixed in the presence of a liquid, typically water that causes the powders to clump up into granules. The granulate is screened and/or milled, dried and then screened and/or milled to the desired particle size. The granulate can then be tabletted or other excipients can be added prior to tableting such as a glidant and or lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well suited to direct compression tableting include microcrystalline cellulose, spray dried lactose, dicalcium phosphate dihydrate and/or colloidal silica. The proper use of these and other excipients in direct compression tableting is known to those in the art with experience and skill in particular formulation challenges of direct compression tableting.

A capsule filling of the invention can comprise any of the aforementioned blends and granulates that were described with reference to tableting, only they are not subjected to a final tableting step.

Yet more particularly, a tablet can, for example, be formulated by blending and directly compressing the composition in a tablet machine.

A capsule can, for example, be prepared by filling half of a gelatin capsule with the above tablet composition and capping it with the other half of the gelatin capsule.

A simple parenteral solution for injection can, for example, be prepared by combining carvedilol of the invention, sterile propylene glycol, and sterile water and sealing the composition in a sterile vial under sterile conditions.

Capsules, tablets and lozenges and other unit dosage forms preferably contain a dosage level of about 1 mg to about 100 mg of carvedilol described herein.

Another embodiment of the present invention provides a method for treating a patient suffering from hypertension, congestive heart failure, or another condition that would benefit from treatment with the carvedilol of the invention, comprising the step of administering to the patient a pharmaceutical composition comprising a therapeutically effective amount of the carvedilol of the invention described herein.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

The following examples are given for the purpose of illustrating the invention and shall not be construed as limiting the scope or spirit of the invention.

### EXAMPLES

### Example 1

### Preparation of Carvedilol Phosphate Free of Bis

20 gr of carvedilol (containing 0.07% and 0.11% of Bis 1 and Bis 2 respectively) were charged into 1 liter glass reactor equipped with mechanical stirrer, and controlled heating/cooling system. 600 ml of EtOH abs (Ethanol absolute) were charged, the agitator was turned on and the Reactor content was heated to reflux (78-82° C.), during the heating full dissolution was achieved.

3.4 ml of 85% phosphoric acid was introduced into the reactor and the reactor content was stirred for 17 hr, cooled to 15° C. and stirred for 2 hours, filtered and washed with 40 ml absolute ethanol.

The cake product was dried in a vacuum oven under a reduced pressure (under 100 mm Hg) at 55° C. until a dried product was obtained.

The resulting solid was analyzed by HPLC and showed carvedilol dihydrogen phosphate containing less than 0.03% (w/w) (quantitation limit) of each diastereomer (Bis 1 and Bis 2).

### Example 2

### Purification of Carvedilol Phosphate from the Bis Impurity

20 g. (on dry basis) of wet carvedilol (containing 0.07% and 0.15% of Bis 1 and Bis 2 respectively) was charged into 1 liter glass reactor equipped with mechanical stirrer, and controlled heating/cooling system. 540 ml of absolute ethanol were added, The agitator was turned on and the Reactor content was heated to reflux (78-82° C.), during the heating full dissolution was achieved.

3.75 ml of 85% phosphoric acid and 60 ml of absolute ethanol were added into the reactor and the reactor content was stirred for 22 hr, during which precipitation was observed, cooled to 15° C., filtered and washed with 40 ml absolute ethanol.

The cake product was dried in a vacuum oven under a reduced pressure (less than 100 mmHg) at 55° C. The resulting solid was analyzed by HPLC and showed carvedilol dihydrogen phosphate containing less than 0.03% (w/w) (quantitation limit) of each diastereomer (Bis 1 and Bis 2).

### Example 3

### Preparation of Carvedilol Base, Starting from Carvedilol Phosphate

26.7 g. of sodium bicarbonate and 240 ml of water were charged into 1 liter glass reactor equipped with mechanical stirrer, and controlled heating/cooling system. The reactor content was heated to 45° C., and 288 ml of ethyl acetate and 48 g. of carvedilol phosphate were added. The reactor content was heated to 55° C. and stirred until full dissolution was observed. After water separation, 100 ml of ethyl acetate were added and the organic phase was washed 2 times with 100 ml of water.

The reactor content was cooled to 25° C., seeded with Carvedilol-base, and stirred for 16 hours until precipitation. The resulting solid was filtered washed with 50 ml ethyl acetate, and dried in a vacuum oven under a reduced pressure (less than 100 mmHg) at 50° C.

### Example 4

### Comparative Example Using Isopropanol Instead of Ethanol

50 g. (on dry basis) of carvedilol (containing 0.09% and 0.12% of Bis 1 and Bis 2 respectively), 500 ml isopropanol, and 8.5 ml of 85% phosphoric acid were charged into a 1 liter glass reactor equipped with a mechanical stirrer and controlled heating/cooling system. The agitator was turned on and the reactor content was heated to reflux (78-82° C.), stirred for 2 hr, cooled to 15° C., filtered and washed with 50 ml isopropanol.

The cake product was dried in a vacuum oven under a reduced pressure (under 100 mmHg) at 55° C. until a dried product was obtained.

The resulting solid was analyzed by XRD and showed carvedilol dihydrogen phosphate Form I content.

The resulting solid was analyzed by HPLC and was found to contain 0.09% Bis 1 and 0.11% Bis2.

### Example 5

### HPLC Method

### HPLC

| Column & | Phenomenex, Luna C8(2), 5μ, 250 × 4.6 mm | | | |
|---|---|---|---|---|
| Packing: | | | | |
| Buffer: | 0.02M KH₂PO₄ | | | |
| Eluent A; | 65% buffer 35% Acetonitrile (gradient grade) | | | |
| Eluent B: | 20% buffer 80% Acetonitrile (gradient grade) | | | |
| | Time | Flow | Eluent A | Eluent B |
| | (min) | (mL/min) | (%) | (%) |
| Gradient | 0 | 0.8 | 100 | 0 |
| | 3 | 0.8 | 100 | 0 |
| | 4 | 1.3 | 100 | 0 |
| | 15 | 1.3 | 100 | 0 |
| | 35 | 1.3 | 0 | 100 |

| | |
|---|---|
| Equilibration | 10 min |
| time: | |
| Sample volume: | 20 μL |
| Detector: | 242 nm |
| Column | 30° C. |
| temperature: | |
| Autosampler | 20° C. |
| temperature | |
| Diluent | Mixture of water and Acetonitrile (65:35 V/V) |

### System Suitability Solution

The marker solution contains 0.3 mg/mL of CRV-P and 0.003 mg/mL of Bis. The retention time of CRV-P peak is about 9.0 minutes. Typical relative retention time is 3.25 for the Bis 1 peak and 3.28 for the Bis 2 peak relative to the CRV-P peak.

### Sample Solution Preparation

Sample solution contains 0.3 mg/mL of CRV-P sample.

### Calculation

Calculation should be produced on a dried basis against 0.1% of CRV-P standard. RRF 1.2 for Bis 1 and Bis 2.

### Formula

a) Calculate the percent amount of Bis 1 and Bis 2 impurity peaks in the sample according to their relative response factor:

$$\% imp = \frac{Area of imp \times potency of CRV - P standard}{\begin{matrix}Average response factor of CRV - \\ P std \times conc . sample \times RRF\end{matrix}}$$

b) Calculate the percent amount of any impurity peaks in the sample according to the following formula:

$$\% imp = \frac{Area of imp \times potency of CRV - P standard}{Average response factor of CRV - P std \times conc . sample}$$

### Abbreviations

CRV-P═(±)-1-(9H-carbazol-4-yloxy)-3-{[2-(2-methoxyphenoxy)ethyl]amino}-2-propanol phosphate salt (1:1)
Diol=4-(ethylenediol methoxy)-9H carbazole
Epoxy (OMC)=4-(2,3-epoxypropoxy)carbazole

What is claimed is:

1. A process for reducing bis impurities ((1,1′-[2-(2-methoxyphenoxy)ethyl]iminobis-[3-(9H-carbazol-4-yloxy)]-propan-2-ol)) in carvedilol comprising:
(a) combining carvedilol base with phosphoric acid in ethanol to obtain a reaction mixture; and
(b) precipitating carvedilol phosphate from the reaction mixture.

2. The process of claim 1 where the carvedilol phosphate of step (b) contains less than about 0.03% (w/w) each diastereomer of the bis impurities.

3. The process of claim 2 where step (a) comprises combining carvedilol base with EtOH, heating, adding phosphoric acid, and cooling.

4. The process of claim 3 where heating is carried out from about room temperature to about reflux temperature.

5. The process of claim 3 where heating is carried out to obtain a solution.

6. The process of claim 3 where heating is carried out to a temperature of about 60° C. to about 80° C.

7. The process of claim 4 where heating is carried out to about reflux temperature.

8. The process of claim 3 where cooling is carried out to a temperature of about 0° C. to about 40° C.

9. The process of claim 3 where cooling is carried out to a temperature of about 10° C. to 30° C.

10. The process of claim 3 where cooling is carried out to about 25° C.

11. The process of claim 1 further comprising drying the carvedilol phosphate.

12. The process of claim 1 further comprising drying at a temperature of about 25 C to 100° C.

13. The process of claim 1 further comprising drying at a temperature of about 55° C. and a pressure of less than about 100 mmHg.

14. The process of claim 1 wherein the carvedilol phosphate is carvedilol dihydrogen phosphate.

15. The process of claim 1 wherein the carvedilol phosphate is carvedilol hydrogen phosphate.

16. The process of claim 1, wherein there is at least about 50% reduction in the amount of the impurities.

17. The process of claim 1, wherein there is at least about 100% reduction in the amount of the impurities.

18. A process for preparing carvedilol base with reduced bis impurities ((1,1′-[2-(2-methoxyphenoxy)ethyl]iminobis-[3-(9H-carbazol-4-yloxy)]-propan-2-ol)) comprising:
(a) combining carvedilol base with phosphoric acid in ethanol to obtain a reaction mixture;
(b) precipitating carvedilol phosphate from the reaction mixture; and
(c) reacting the carvedilol phosphate of step (b) with a base.

19. The process of claim 18, wherein the base is sodium or potassium carbonate or hydroxide.

20. The process of claim 18, wherein the base is sodium bicarbonate.

21. A process for obtaining carvedilol free of bis impurities ((1,1′-[2-(2-methoxyphenoxy)ethyl]iminobis-[3-(9H-carbazol-4-yloxy)]-propan-2-ol)) comprising:
(a) combining carvedilol base with phosphoric acid in ethanol to obtain a reaction mixture; and
(b) precipitating carvedilol phosphate free of bis impurities from the reaction mixture.

22. A preparation of carvedilol base or carvedilol phosphate comprising less than about 0.03% (w/w) each diastereomer of bis impurities.

23. A pharmaceutical composition comprising the preparation of carvedilol base or carvedilol phosphate of claim 22 and a pharmaceutically acceptable excipient.
